(19) United States
(12) Patent Application Publication
(10) Pub. No.: US 2016/0367605 A1
(43) Pub. Date: Dec. 22, 2016

(54) HEALING COMPOSITION AND USE THEREOF

(71) Applicant: MELIPHARM, Limoges (FR)

(72) Inventors: Fabien QUERO, LE BOUSCAT (FR); Carmen MALEPEYRE, VERNEUIL SUR VIENNE (FR); Anais RAYNAUD, LE PALAIS SUR VIENNE (FR); Jean-Michel PETIT, ISLE (FR)

(73) Assignee: MELIPHARM, Limoges (FR)

(21) Appl. No.: 14/900,504

(22) PCT Filed: May 14, 2014
(86) PCT No.: PCT/FR2014/051123

§ 371 Date: Dec. 21, 2015

(30) Foreign Application Priority Data
Jun. 20, 2013 (FR) 1355810

(51) Int. Cl. A61K 35/644; A61K 9/06; A61K 31/716; A61K 38/39; A61K 9/00; A61K 31/728

(52) U.S. Cl. CPC A61K 35/644; A61K 9/0014; A61K 31/728; A61K 31/716; A61K 38/39; A61K 9/06

(57) ABSTRACT

A healing and/or antibacterial composition in particular comprising honey, for use as a topical healthcare product on the skin or mucous membranes.

### FIELD OF INVENTION

The present invention relates to a particular composition made up of honey and at least one component of the extracellular matrix and/or at least one beta-glucan, and its use on the skin or mucous membranes for healing and/or antibacterial activity.

### BACKGROUND OF INVENTION

A wound is a rupture of the cutaneous barrier which, aside from the lesion of underlying organs, may cause infectious agents to penetrate the body. The human and animal tissues are capable of repairing a wound through their own unique repair processes, and healing is a natural biological phenomenon. This ability nevertheless remains subject to many variations, the speed and quality of the healing of the wound depending on the general condition of the affected body, the condition and location of the wound and the occurrence or absence of an infection.

Consequently, in case of cutaneous lesion, it is necessary to apply products promoting healing. Several healing products currently exist.

Some of these healing products act by maintaining a moist environment to promote healing, for example hydrocolloids made up of absorbing polymers and other gelling agents, alginates, made up of calcium and sodium alginate, hydrogels, absorbing dressings or dressings with hyaluronic acid. Although these products promote healing by creating a moist environment, they do not have antibacterial properties. Alternatives of these silver- or iodine-based have therefore been developed to fill this gap. However, although they are highly antibacterial, silver dressings often have the drawback of being cytotoxic. They cause cell death in the cells involved in healing of the wounds. It is consequently not recommended to use silver-based dressings over long periods of time due to their cytotoxicity. Iodine-based products are also cytotoxic, and their antimicrobial activity decreases considerably in the presence of organic materials (pus, fibrin or necrosis). Furthermore, there is a risk of sensitivity in contact with eczema and allergy to these products.

### SUMMARY OF THE INVENTION

That is why the present invention aims to propose a honey-based composition offsetting the drawbacks of the prior art that is easy to manufacture, cost-effective and easy to apply, and that creates both a moist environment and antibacterial activity in order to unite the conditions favorable to quick and effective healing without creating the toxicity of the aforementioned products.

To that end, the invention relates to a composition comprising a mixture of honey and at least one component of the extracellular matrix chosen from among collagen, elastin and glycosaminoglycans, in particular hyaluronic acid and/or at least one beta-glucan, in particular for use thereof as a healing and/or antibacterial topical healthcare product.

The use of wrappings and care involving honey to treat certain wound infections is known. It is in particular known that honey has antibacterial and antifungal properties, and that it makes it possible to reduce healing times without infections or side effects.

However, the mixture according to the invention has a significant and surprising synergistic effect, which makes it possible to propose a healing, reparative and antibacterial composition with good tolerance and with no noticeable side effects, and with properties exceeding those of the products currently available.

Other features and advantages will emerge from the following detailed description of the invention.

### DETAILED DESCRIPTION OF THE INVENTION

The invention therefore relates to a composition to be used as a healing and/or antibacterial topical healthcare product on the skin or mucus membranes, comprising honey and at least one component of the extracellular matrix chosen from among collagen, elastin and glycosaminoglycans, in particular hyaluronic acid and/or at least one beta-glucan.

Within the meaning of the invention, honey refers to a natural or artificial honey, or a mixture of several natural and/or artificial honeys.

“Artificial honey” refers to at least one sugar. It may also be a combination of at least two sugars.

Preferably, the honey is a natural honey chosen from among thyme, honeydew, buckwheat and manuka honeys, and mixtures thereof. It may for example be a honey as described in application FR 1,258,722.

If the honey used in the composition is an artificial honey, it is preferably a saccharide mixture primarily comprising glucose and/or fructose and/or saccharose.

In addition to honey, the composition according to the invention comprises:
at least one component of the extracellular matrix chosen from among collagen, elastin and glycosaminoglycans such as hyaluronic acid, heparan sulfates, keratan sulfates or chondroitin sulfates, and/or
at least one beta-glucan.

The beta-glucans present in the composition are polysaccharides made up of glucose units connected by β(1,3) and/or β(1,4) and/or β(1,6) bonds.

These may for example be beta-glucans with a molecular weight comprised between 0.5, 10⁶ Da and 1.10⁶ Da.

The glycosaminoglycans present in the composition may be chosen from among hyaluronic acid, heparan sulfates, keratan sulfates and/or chondroitin sulfates.

The hyaluronic acid present in the composition is preferably a hyaluronic acid salt or a hyaluronic acid derivative, still more preferably a hyaluronic acid sodium salt with a molecular weight greater than 20 kDa.

According to one suitable embodiment, the hyaluronic acid present in the composition has a molecular weight greater than 100,000 Da.

The collagen present in the composition is preferably a collagen peptide from the fish, porcine or bovine collagen hydrolysate or a collagen biomimetic peptide.

The elastin present in the composition is preferably a cattle tendon hydrolysate or an elastin biomimetic.

According to one particularly suitable embodiment, the composition comprises between 5 wt % and 99.8 wt % of honey in dry matter of the composition.

If the composition comprises hyaluronic acid, it is present between 0.05 wt % and 10 wt % of dry matter of the composition, still more preferably between 0.05% and 1%.

If the composition comprises collagen, it is present between 0.001 wt % and 50 wt % of dry matter of the composition, still more preferably between 0.001% and 5%.

If the composition comprises elastin, it is present between 1 wt % and 25 wt % of the dry matter of the composition, still more preferably between 5% and 10%.

If the composition comprises a glycosaminoglycan other than hyaluronic acid, it is present between 0.1 wt % and 20 wt % of the dry matter of the composition, still more preferably between 0.1% and 10%.

If the composition comprises a beta-glucan, is present between 0.01 wt % and 10 wt % of the dry matter of the composition, still more preferably between 0.01% and 5%.

The composition may be made up exclusively of:
honey, and
collagen and/or elastin and/or glycosaminoglycans (in particular hyaluronic acid) and/or beta-glucan, or a mixture thereof.

It may also additionally contain lactoferrin, glucose oxidase, zinc oxide, lactoperoxidase, thiocyanates, lysozyme, xylitol, vanillin, sugars such as galactose, dextrose, rhamnose, mannose, teflose®, oligosaccharides, lactobionic acid, bisabolol, allantoin, aloe vera, propolis, squalane, vitamins, panthenol, ceramides, bentonite and/or kaolin.

It may also contain excipients chosen from among excipients that are dermatologically compatible and/or applicable on the skin and mucous membranes in order to obtain a composition in powder form, or in liquid form such as a lotion, or semi-solid such as a cream, pomade, spray, gel, paste, suppository, vaginal suppository or powder. These may for example be excipients such as sugar and sugar derivatives, polysaccharides (pectin, starch and derivatives, alginate and derivatives, chitosan and derivatives, cellulose derivatives, gums), synthesis polymers, waxes, natural or artificial oils, butters, waxes, mineral products (silica, talc, clays, titanium oxide), glycerides and other fatty esters, surface active agents, water, ethanol, propylene glycol, butylene glycol, polyethylene glycol, glycerol, sorbitol, hydrocarbons and silicones, proteins and peptides, and other excipients known by those skilled in the art.

The composition may also comprise auxiliary formulation additives such as surface active agents, gelling agents, absorbent agents, humectants, solvents, spreading agents, stabilizers, sequestering agents, rheological modifiers, preservatives, antioxidants and antimicrobials, dyes and scents.

According to one particularly suitable alternative of the invention, in addition to honey, the composition further comprises at least one hyaluronic acid and at least one beta-glucan.

The composition according to the invention may be sterile, i.e., it has undergone a sterilization process. The sterilization is preferably done by gamma radiation.

The composition according to the invention may be obtained by simple mixing of the components.

Preferably, if the composition contains:
a glycosaminoglycan: it is obtained by the dispersion of a powder (for example, a fine hyaluronic acid powder) or a solution (for example, a hyaluronic acid solution) in a honey composition,
a beta-glucan: it is obtained by dispersion of the beta-glucan in a honey composition either in powdered form or in the form of a beta-glucan solution,
collagen: it is obtained by adding collagen in a honey composition in the form of a powder or in the form of a solution,
elastin: it is obtained by dispersion of an elastin solution in a honey composition.

If the method is a hot method, the heating temperature of the honey or the mixture containing honey must be below 40° C.

The composition according to the invention may assume the form of a powder or various liquid or semi-liquid forms, in particular lotion, cream, emulsion, gel, pomade, spray, vaginal suppository, suppositories.

The composition is used as a healing and/or antibacterial product. In fact, the mixture of honey and beta-glucan and/or component of the extracellular matrix acts synergistically to both:
increase skin cell proliferation and migration, in particular for fibroblasts and keratinocytes,
decrease the bacterial load, and thereby indirectly promote healing.

The effects obtained are much better than those obtained with honey alone or beta-glucans alone or a component of the extracellular matrix alone. There is a surprising synergistic effect. At the cellular level, honey alone or beta-glucans alone or components of the extracellular matrix alone make it possible to increase the proliferation of fibroblasts and keratinocytes, but when they are used together, the increased proliferation of fibroblasts and keratinocytes is greater than the sum of the proliferations caused by the components alone (see in particular the test results in points 3 to 6). Furthermore, at the bacterial level, neither the hyaluronic acid, nor the beta-glucans, nor honey, when they are greatly diluted, have an antibacterial activity. However, the combination of the components according to the invention, in particular the combination of hyaluronic acid with honey or of beta-glucan with honey, or hyaluronic acid and beta-glucan with honey, at the same concentration, shows a significant inhibition of bacterial proliferation (see table 1).

According to the invention, the composition may therefore advantageously be used as a healthcare product for topical application on the skin or mucous membranes in humans or animals, very preferably as:
medical device designed to be applied on irritations or skin lesions, in particular to:
   treat first and second degree burns,
   treat and/or prevent postoperative healing disunions,
   treat and/or prevent postoperative residual cavities of the pilonidal sinuses,
   treat surgical scars infected after flattening,
   treat ulcers and eschars,
   treat traumatic and/or surgical wounds,
   treat acute and chronic wounds,
   treat cancerous wounds,
   treat ostomy locations,
   treat dermabrasions,
   treat superficial wounds,
a composition, for example a medical device or a dermatological composition, for the treatment of dermatitis, acne or diaper rash, impetigo, folliculitis, boils, whitlow, mycosis,
oral gel, in particular to treat canker sores and mucositis,
lip stick or balm designed to treat cracking of the skin,
cream for the hands and feet, in particular designed to treat cracks and chilblains,
balm intended for the prevention, care and/or treatment of cracked nipples,
vaginal gel,
rectal gel, suppository or rectal cream.

A medical device refers to a device intended to be used in humans or animals, in particular for the prevention, control, treatment and/or attenuation of a disease or injury.

If it is a medical device for healing skin lesions in the form of a powder, spray, gel, cream or pomade, the composition according to the invention may in particular apply according to the following usage protocol:
rinse the wound and dry gently,
cover the entire wound or the secondary dressing with a film of the composition according to the invention,
cover with compresses and occlusive dressing during first applications for very wet wounds.

During the cleaning phase, and depending on the condition of the wound, it is advised to apply a new dressing one to two times every 24 hours.

During the budding phase, it is advised to apply a new wrapping every 48 to 72 hours.

During the epithelialization phase, it is recommended to apply a new dressing every 3 or 4 days.

The invention will now be illustrated through example compositions and results from trials demonstrating its efficacy.

A. Example Compositions According to the Invention

### 1. EXAMPLE 1

The composition of example 1 is a hyperosmotic gel. It is made up of:
99.8% of a mixture of thyme, honeydew, buckwheat and manuka honeys,
0.2% sodium hyaluronate,
   the percentages being given in weight percentages relative to the total weight of the dry mass of the composition.

The composition is obtained by mixing two compounds.

### 2. EXAMPLE 2

The composition of example 2 is a pomade made up of:
quantity sufficient for 100% thyme honey,
25% triglycerides,
11% beeswax,
7.5% shea butter,
1.83% tocopheryl acetate,
5% glyceryl stearate,
0.2% sodium hyaluronate,
0.07% tocopherol,
   the percentages being weight percentages relative to the total weight of the dry mass of the composition.

The composition is obtained by carrying out the following steps:
finely dispersing the sodium hyaluronate in the honey,
heating the mixture to less than 40° C.,
heating the lipophilic phase (wax, butter and glycerides) to approximately 70° C. to melt the solids,
when the lipophilic phase has reached the temperature of 40° C., adding the tocopheryl acetate and the mixture of honey and sodium hyaluronate,
mixing the pomade until it cools.

### 3. EXAMPLE 3

The composition of example 3 is a cream made up of:
quantity sufficient for 100% artificial honey,
30% glycerol,
10.3% shea butter,
5% cetearyl alcohol and cetearyl glucoside,
2% white beeswax,
1.82% tocopheryl acetate,
0.8% stearic acid,
0.2% sodium hyaluronate,
0.07% tocopherol,
0.01% ascorbyl palmitate,
   the percentages being weight percentages relative to the total weight of the dry mass of the composition.

The composition is obtained by carrying out the following steps:
finely dispersing the sodium hyaluronate in the honey,
heating the mixture to less than 40° C.,
heating the lipophilic phase (wax, butter) and the glycerol separately to approximately 70° C.,
adding the lipophilic phase to the glycerol and, when that mixture has reached about 40° C., adding the tocopheryl acetate, the tocopherol, the ascorbyl palmitate, then the mixture of honey and sodium hyaluronate,
mixing the cream until it cools.

### 4. EXAMPLE 4

The composition of example 4 is a hyperosmotic gel. It is made up of:
90% buckwheat honey,
9.8% glycerol,
0.2% sodium hyaluronate,
   the percentages being weight percentages relative to the total weight of the dry mass of the composition.

The composition is obtained by carrying out the following steps:
a solution of 2% sodium hyaluronate in a water/glycerol/ethanol mixture (in a 10/10/3 ratio) is prepared, then dehydrated by vacuum distillation;
this sodium hyaluronate glycerite solution is next mixed into the honey at ambient temperature.

### 5. EXAMPLE 5

The composition of example 5 is a hyperosmotic gel. It is made up of:
94.8% of a mixture of thyme, honeydew, buckwheat and manuka honeys,
5% of a fish collagen,
0.2% sodium hyaluronate,
   the percentages being weight percentages relative to the total weight of the dry mass of the composition.

The composition is obtained by mixing the three compounds.

### 6. EXAMPLE 6

The composition of example 6 is a hyperosmotic gel. It is made up of:
95% of a mixture of thyme, honeydew, buckwheat and manuka honeys,
5% of a beta-glucan solution,
   the percentages being weight percentages relative to the total weight of the dry mass of the composition.

The composition is obtained by mixing the two compounds.

### 7. EXAMPLE 7

The composition of example 7 is a hyperosmotic gel. It is made up of:
94.8% of a mixture of thyme, honeydew, buckwheat and manuka honeys,
5% of a beta-glucan solution,
0.2% sodium hyaluronate,
   the percentages being weight percentages relative to the total weight of the dry mass of the composition.

The composition is obtained by mixing the three compounds.

B. Evaluation of the Efficacy of the Composition According to the Invention

Trials have been conducted to demonstrate the healing and antibacterial efficacy of several compositions according to the invention and to compare that efficacy to that of the components alone.

1. Evaluation of the Antibacterial Efficacy of the Composition According to the Invention Relative to the Ingredients Alone

The aim of this study is to evaluate the antibacterial activity of the composition according to the invention (examples 1, 6 and 7), compared to that of honey alone. The specimens were tested at 3, 5, 7 and 9% v/v.

The operating protocol is as follows.

The bacterial proliferation in the presence of the products to be tested is determined using a 96 well microplate method. Each well is inoculated with 50 μL of bacterial suspension to be tested in a Muller-Hinton (MH) broth+150 μL of diluted test product solution. The bacterial concentration in the well is set at 10⁶ CFU/mL. The positive control (150 μL of bacterial suspension+50 μL of MH medium) corresponds to 100% bacterial proliferation. The negative control (200 μL of culture medium) corresponds to 0% bacterial proliferation. Each specimen is tested in triplicate. The optical densities (OD) are read a first time at time 0 (T0) at 450 nm. The plates are then incubated for 24 h at 37° C. with agitation. At the end of incubation, the OD is measured again at 450 nm (T24). The proliferation percentage is calculated as follows:

Proliferation=(DO_{T24}−DO_{T0})/(DO_{T24 Control +}−DO_{T0 Control +})

The obtained results in bacterial proliferation percentage are shown in table 1 below.

TABLE 1
Evaluation of antibacterial activity

| | | Bacterial proliferation |
|---|---|---|
| | Tested concentration | percentage |
| | (in % v/v) | (Staphylococcus aureus) |
| Honey alone | 3 | 32% |
| | 5 | 25% |
| | 7 | 19% |
| | 9 | 15% |
| Composition | 3 | 25% |
| example 1 | 5 | 15% |
| | 7 | 13% |
| | 9 | 1% |
| Composition | 3 | 28% |
| example 6 | 5 | 23% |
| | 7 | 11% |
| | 9 | 2% |
| Composition | 3 | 27% |
| example 7 | 5 | 20% |
| | 7 | 13% |
| | 9 | 6% |

One can see that the compositions according to the invention have improved antibacterial activity. Furthermore, this activity is more significant than that of honey alone.

2. Evaluation of the Healing Activity-Cell Proliferation of Hyaluronic Acid

The aim of this study is to evaluate the healing activity of a composition according to the invention, compared to that of honey alone and that of hyaluronic acid alone, and to verify the stability of the composition after sterilization with gamma radiation.

The tested specimens are as follows:
raw honey: thyme honey,
hyaluronic acid tested alone at 0.5%,
composition made up of raw thyme honey to which 0.2% or 0.5% of hyaluronic acid was added,
composition made up of raw thyme honey to which 0.2% of hyaluronic acid was added, and treated by gamma radiation (30 kGy).

The products are diluted to obtain a concentration of 5.12% v/v of the specimen.

The operating protocol is as follows:

The primary cultures of PAR 08052 human fibroblasts are next seeded in 48 well microplates at a rate of 10,000 cells per well with a whole culture medium volume of 500 μL. The cells will next adhere to the bottom of the wells for 24 hours. The medium will next be replaced by the composition according to the invention diluted in the culture medium without serum. The tested concentration is 1.3% v/v at a rate of 500 μL per well and incubated for 48 hours at 37° C.+5% CO₂. The cells are next taken off with trypsin, then counted on Malassez slides with an exclusion dye (Trypan blue). The results are indicated in number of cells per well. The obtained results are shown in table 2 below.

TABLE 2
Evaluation of healing activity

| | Number of cells per well |
|---|---|
| Honey alone | 13,333.3 |
| Hyaluronic acid 0.5% | 15,833.3 |
| Honey + Hyaluronic acid 0.2% | 20,833.3 |
| Honey + Hyaluronic acid 0.5% | 20,000 |
| Honey + Hyaluronic acid 0.2% + | 20,000 |
| gamma radiation | |

One can see that the composition according to the invention promotes the proliferation of fibroblasts, and therefore healing, and that this effect is significantly improved compared to that of honey alone or that of hyaluronic acid alone.

Furthermore, this activity is maintained even after gamma ray sterilization.

3. Evaluation of the Synergistic Effect of a Honey+Hyaluronic Acid Mixture

The aim of these trials was to demonstrate the synergistic effect of the mixture according to the invention.

The fibroblast proliferation percentage is determined relative to a control not treated with the composition.

a) Berenbaum Test

The Berenbaum test makes it possible to determine the additive, synergistic or antagonistic effects between two products placed in mixtures in contact with cells (Berenbaum, 1977). This test was developed in order to determine the impact of several mixed compounds using experimental data inserted into a mathematical formula. It is applicable to many areas of biology.

This test is applied here to honey and hyaluronic acid (HA). To that end, HA and honey are used to stimulate the fibroblasts for 48 h. The honey and HA are placed in the culture medium at concentrations below the stimulation maximum. After 48 h of stimulation, the cells are counted and the proliferation percentage is calculated.

The results of the counts are analyzed based on a search for equivalent activity for each compound. For example, the activity sought is a proliferation of 150%. The dose of honey necessary to obtain 150% proliferation (in the case of our example) is designated by Aeq for honey. Beq designates the equivalent dose necessary to obtain the same effect (150% proliferation) with HA. This search for equivalent concentrations is done owing to the production of the line of honey alone and the line of HA alone.

Secondly, the mixture of the two compounds makes it possible to determine the doses of each product to be introduced together to obtain the same effect (150% proliferation) as with Aeq or Beq. The necessary dose of honey is then designated as A, and that of the HA as dose B.

Subsequently, it is possible to introduce this data into the following equation:

$$\frac{Dose A}{Aeq} + \frac{Dose B}{Beq}$$

If the result of this equation is equal to 1, the effect of the 2 compounds will be additive. If the result is less than 1, synergy exists. However, if the result is greater than 1, the compounds are designated antagonistic.

b) Production of a Hyaluronic Acid (HA) Line

Treatment duration=48 h

Cells tested=fibroblasts

The tested concentrations correspond to the hyaluronic acid concentrations found in the culture wells once the example products have been diluted. These dilutions are necessary to carry out this test.

TABLE 3

| | | | | | | | | |
|---|---|---|---|---|---|---|---|---|
| Quantity of | 0% | 0.00023% | 0.00047% | 0.00094% | 0.0018% | 0.0037% | 0.0075% | 0.015% |
| hyaluronic | | | | | | | | |
| acid (%) | | | | | | | | |
| Proliferation | 100% | 100% | 109% | 127% | 182% | 182% | 182% | 190% |
| percentage (%) | | | | | | | | |

c) Production of a Thyme Honey Line

Treatment duration=48 h

Cells tested=fibroblasts

TABLE 4

| | | | | | |
|---|---|---|---|---|---|
| Quantity of honey (%) | 0% | 0.3% | 0.6% | 1.28% | 2.56% |
| Proliferation percentage (%) | 100% | 138% | 145% | 154% | 163% |

d) Production of Thyme Honey+HA Mixtures

Treatment duration=48 h

Cells tested=fibroblasts

TABLE 5
Overview of the obtained results and calculation of concentrations to be used
in the composition - Overview of concentrations with synergistic effects

| | Calculated HA | | | | |
|---|---|---|---|---|---|
| | quantity, in the | | | | |
| Tested | composition with a | Honey | | Honey + | |
| composition | honey quantity of | (approximately 100%) | Hyaluronic acid | hyaluronic acid | Berenbaum |
| percentages | approximately 100% | (proliferation %) | (proliferation %) | (proliferation %) | index |
| Tested at 0.6% | 0.05% | 145.5% | 100% | 173% | 0.36 |
| Tested at 0.30% | 0.1% | 138.2% | 100% | 167% | 0.24 |
| Tested at 0.3% | 0.2% | 138.2% | 109% | 164% | 0.28 |
| Tested at 0.6% | 0.2% | 145.5% | 127% | 180% | 0.75 |

For each of the compositions comprising between 0.05% and 0.2% of hyaluronic acid, the Berenbaum index is less than 1. This result indicates a synergy between the honey and the hyaluronic acid causing an increase in fibroblast proliferation at those concentrations.

e) Results on the Stimulation of Collagen III Synthesis

Collagen III is one of the majority compounds of the extracellular matrix. Collagen provides the fibrillar structure of the extracellular matrix allowing mechanical maintenance of the cell tissue.

TABLE 6
Overview of the results obtained on collagen III assay (by ELISA-
type assay) in the fibroblast culture supernatant having been treated
with the various formulas for 48 h. The results are expressed in
relative quantity with respect to the untreated control.

| | | Relative quantity of |
|---|---|---|
| | Relative quantity of | Collagen III assayed in |
| Relative quantity of | Collagen III assayed in | the culture supernatant |
| Collagen III assayed in | the culture supernatant | treated with honey + |
| the untreated control | treated with honey alone | HA |
| 100% | 106% | 125% |

The effects observed with honey and hyaluronic acid on the production of collagen III are greater than the effects caused by honey alone. A beneficial effect of this composition is observed on the production of collagen III by the fibroblasts.

4. Evaluation of the Synergistic Effect of a Honey+Collagen Mixture

a. Fibroblast Proliferation

Type of collagen tested=bovine collagen (dose used in the composition: 5%)

Tested composition concentration=1.28% v/v

Treatment duration=48 h

Cells tested=fibroblasts

TABLE 7

| | | | Collagen + |
|---|---|---|---|
| Specimens | Honey | Collagen | honey |
| Fibroblast proliferation | 188% | 172% | 261% |
| percentage (%) | | | |

A synergistic effect of the honey and collagen is observed on fibroblast proliferation.

b. Principle of the Proliferation Tests on Keratinocytes

The products are diluted to obtain a concentration of 0.1% v/v of the specimen.

The operating protocol is as follows:

A keratinocyte cell line is seeded in 48 well microplates at a rate of 20,000 cells per well with a whole culture medium volume (10% serum) of 500 μL. The cells will next adhere to the bottom of the wells for 24 hours. The medium will next be replaced by the product to be tested, diluted at 0.1% in the culture medium containing 2.5% serum. The product is tested at a rate of 500 μL per well and incubated for 24 hours at 37° C.+5% CO₂. The cells are next taken off with trypsin, then counted on Malassez slides with an exclusion dye (Trypan blue). The keratinocyte proliferation percentage is determined relative to a control not treated with the specimens.

c. Keratinocyte Proliferation

Type of collagen tested=collagen peptide; dose used in the composition: 1.5%

Tested composition concentration=0.1% v/v

Treatment duration=24 h

Cells tested=keratinocytes

TABLE 8

| | | | Collagen + |
|---|---|---|---|
| Specimens | Honey | Collagen | honey |
| Keratinocyte proliferation | 143% | 129% | 186% |
| percentage (%) | | | |

A synergistic effect of the honey and collagen combination is observed on keratinocyte proliferation.

5. Evaluation of the Synergistic Effect of a Honey+Beta-Glucan Mixture

a) Keratinocyte Proliferation

Tested composition concentration=0.1% v/v

Treatment duration=24 h

Cells tested=keratinocytes

TABLE 9

| | | Beta- | Honey + Beta- |
|---|---|---|---|
| Specimens | Honey | glucans | glucans |
| Keratinocyte proliferation | 131% | 162% | 193% |
| percentage (%) | | | |

A beneficial effect of the honey and beta-glucan combination is observed on keratinocyte proliferation.

b) Results on the Stimulation of Collagen III Synthesis

Collagen III is one of the majority compounds of the cellular matrix. Collagen provides the fibrillar structure of the extracellular matrix allowing mechanical maintenance of the cell tissue.

TABLE 10
overview of the results obtained on collagen III assay
(by ELISA-type assay) in the fibroblast culture supernatant having
been treated with the various formulas for 48 h. The results are
expressed in relative quantity with respect to the untreated control.
Relative quantity of Collagen III assayed
in the culture supernatants treated with

| Control | Honey | Beta- | Honey + Beta- |
|---|---|---|---|
| not treated | alone | glucans | glucans |
| 100% | 106% | 88% | 119% |

The quantity of collagen III produced by the fibroblasts is noticeably higher than that obtained with honey alone or beta-glucans alone. This result indicates that the honey mixed with beta-glucans has a synergistic effect on the production of collagen III by the fibroblasts.

6. Evaluation of the Synergistic Effect of a Honey+Hyaluronic Acid+Beta-Glucan Mixture

Tested specimen concentration=1.28% v/v

Treatment duration=48 h

Cells tested=fibroblasts

TABLE 11

| | | | | | Honey + |
|---|---|---|---|---|---|
| | | | | Honey + | hyaluronic |
| | | Hyaluronic | Beta- | Beta- | acid + |
| Specimens | Honey | acid | glucans | glucans | Beta-glucans |
| Fibroblast | 154 | 182 | 222 | 189 | 239 |
| proliferation | | | | | |
| percentage | | | | | |
| (%) | | | | | |

The honey+hyaluronic acid+beta-glucan combination causes an increase in fibroblast proliferation relative to the compounds alone.

1. A composition comprising:
honey, and
at least one component of the extracellular matrix chosen from among collagen, elastin and glycosaminoglycans and/or at least one beta-glucan,
for use as a topical healing and/or antibacterial healthcare product.

2. The composition according to claim 1, wherein the at least one component of the extracellular matrix is a hyaluronic acid.

3. The composition according to claim 1, wherein the at least one component of the extracellular matrix is a glycosaminoglycan selected from the group consisting of heparan sulfates, keratan sulfates and chondroitin sulfates.

4. The composition according to claim 1, comprising between 5% and 99.8% of honey.

5. The composition according to claim 1, wherein the honey is natural honey.

6. The composition according to claim 5, wherein the honey is selected from the group consisting of thyme honey, honeydew honey, buckwheat honey, manuka honey, and mixtures thereof.

7. The composition according to claim 1, wherein the honey is artificial honey.

8. The composition according to claim 7, wherein the artificial honey is primarily made up of glucose and/or fructose and/or saccharose.

9. The composition according to claim 1, wherein the composition is a powder, liquid or semi-liquid.

10. The composition according to claim 1, wherein the composition is in a form of a powder gel, pomade, spray, cream, emulsion or vaginal suppository.

11. The composition according to claim 1, wherein the composition has been treated by gamma radiation.

12. A method of treating a wound, comprising administering to a subject in need thereof an effective amount of a medical device comprising an effective amount of the composition according to claim 1.

13. A method of treating an irritation or skin lesion, comprising applying on a subject in need thereof an effective amount of a medical device comprising the composition according to claim 1, wherein the medical device is applied to treat an irritation or skin lesion selected from the group consisting of first and second degree burns, post-operative scar disunions, residual cavities of the pilonidal cavities, surgical scars infected after flattening, ulcers and eschars, traumatic wounds, acute and chronic wounds, cancerous wounds, ostomy locations, dermabrasions, superficial wounds, and combinations thereof.

14. A method for treating dermatitis, acne, diaper rash, impetigo, boils, whitlow or mycosis, comprising applying to a subject in need thereof an effective amount of the composition according to claim 1, wherein the composition is selected from the group consisting of an oral gel to treat canker sores and mucositis, a lip stick or balm to treat cracked skin, a cream for the hands and feet, balm intended treat cracked nipples, vaginal gel or suppository and a rectal cream.

15. The composition according to claim 1, comprising between 0.05% and 10% of hyaluronic acid by weight of dry matter.

16. The composition according to claim 1, comprising between 0.05% and 1% of hyaluronic acid by weight of dry matter.

17. The composition according to claim 1, comprising hyaluronic acid with a molecular weight greater than 100,000 Da.

18. The composition according to claim 1, comprising hyaluronic acid and at least one beta-glucan.

19. The composition according to claim 1, comprising beta-glucans made up of glucose units connected by a bond selected from the group consisting of β(1,3), β(1,4), and β(1,6) bonds.

20. The composition according to claim 1, comprising between 0.01% and 5% of beta-glucans by weight of dry matter.

21. The composition according to claim 1, comprising between 0.001% and 50% of collagen by weight of dry matter.
